(19) United States
(12) Patent Application Publication
(10) Pub. No.: US 2015/0112214 A1
(43) Pub. Date: Apr. 23, 2015

(54) BLOOD PRESSURE MEASURING DEVICE AND BLOOD PRESSURE MEASURING METHOD

(71) Applicant: SEIKO EPSON CORPORATION, Tokyo (JP)

(72) Inventor: Hiromitsu MIZUKAMI, Shiojiri-shi (JP)

(21) Appl. No.: 14/508,157

(22) Filed: Oct. 7, 2014

(30) Foreign Application Priority Data
Oct. 23, 2013 (JP) 2013-219893
Jul. 25, 2014 (JP) 2014-151559

(51) Int. Cl. A61B 5/02; A61B 5/022

(52) U.S. Cl. CPC A61B 5/02007; A61B 5/022
USPC 600/490

(57) ABSTRACT

A blood pressure measuring device includes: a blood vessel diameter measurement unit which measures a blood vessel diameter of a measurement target blood vessel of a subject; a pressurizing-type sphygmomanometer which acquires a blood pressure of the subject; a calculation unit which calculates a correlation between the blood vessel diameter and the blood pressure on the basis of a result of the measurement by the blood vessel diameter measurement unit and a result of the measurement by the pressurizing-type sphygmomanometer at plural timings during artificial dialysis on the subject with different blood pressure values; and a blood pressure calculation unit which calculates a blood pressure on the basis of the blood vessel diameter of the blood vessel measured by the blood vessel diameter measurement unit, using the correlation, after the artificial dialysis.

### BACKGROUND

1. Technical Field

The present invention relates to a blood pressure measuring device or the like for measuring blood pressures of a subject.

2. Related Art

According to the related art, a technique for measuring blood pressures of a subject is known in which the diameter of a blood vessel (blood vessel diameter) is measured noninvasively so as to find blood pressures in an estimating manner. For example, Motoaki Sugawara, “Development of Method for Noninvasive Measurement of Blood Pressure Waveform,” Medical Electronics and Biological Engineering, 1983, Vol 0.21, p. 429, focuses on the fact that change in blood pressure and change in blood vessel diameter have an approximately linear relation. In the technique disclosed in this literature, change in blood vessel diameter at a measurement site is measured by an ultrasonic echo-tracking method while a maximum blood pressure and a minimum blood pressure are measured with a pressurizing-type sphygmomanometer (cuff-type sphygmomanometer), then a maximum blood vessel diameter is calculated on the basis of the maximum blood pressure, a minimum blood vessel diameter is calculated on the basis of the minimum blood pressure, and thus the change in blood vessel diameter is regarded as a blood pressure waveform. Also, JP-A-2004-41382 discloses a method in which the relation between change in blood pressure and change in blood vessel diameter is regarded as a nonlinear function so that blood pressures are calculated on the basis of a stiffness parameter β indicating the stiffness of the blood vessel, and the blood vessel diameter.

Meanwhile, in medical settings and research settings, there are cases where measurement of blood pressure is monitored continuously for several hours or for several days, or carried out intermittently every several ten minutes or the like. For example, there are cases where the blood vessel diameter is measured continuously or intermittently so as to monitor blood pressures in order to observe the influence of a test drug on cardiac functions or the like. Calculations are necessary in order to maintain measurement accuracy not only in a single or one-off measurement but also in such measurements over relatively long periods.

However, the calculations themselves need to be highly accurate.

### SUMMARY

An advantage of some aspects of the invention is that a technique for realizing highly accurate calculations is proposed.

A first aspect of the invention is directed to a blood pressure measuring device including: a blood vessel diameter measurement unit which measures a blood vessel diameter of a blood vessel; a blood pressure acquisition unit which acquires a blood pressure in the blood vessel; a calculation unit which calculates a correlation between the blood vessel diameter of the blood vessel and the blood pressure in the blood vessel on the basis of a result of the measurement by the blood vessel diameter measurement unit and a result of the acquisition by the blood pressure acquisition unit; and a blood pressure calculation unit which calculates a blood pressure in the blood vessel on the basis of the blood vessel diameter of the blood vessel measured by the blood vessel diameter measurement unit, using the correlation.

As another aspect of the invention, the invention may be configured as a blood pressure measuring method including: carrying out measurement of a blood vessel diameter of a blood vessel and acquisition of a blood pressure in the blood vessel; calculating a correlation between the blood vessel diameter of the blood vessel and the blood pressure in the blood vessel on the basis of a result of the measurement of the blood vessel diameter and a result of the acquisition of the blood pressure; and calculating a blood pressure in the blood vessel on the basis of the result of the measurement of the blood vessel diameter, using the correlation.

According to the first aspect and the like of the invention, the correlation between the blood vessel diameter of a blood vessel and the blood pressure in the blood vessel can be calculated on the basis of the result of the measurement of the blood vessel diameter and the result of the acquisition of the blood pressure. Calculating the correlation based on plural data that are significant for the calculation of the correlation enables realization of a highly accurate calculation. As a matter of course, after the correlation between the blood vessel diameter and the blood pressure is calculated, the blood pressure can be calculated on the basis of the result of the measurement of the blood vessel diameter, without acquiring the blood pressure.

A second aspect of the invention is directed to the blood pressure measuring device according to the first aspect of the invention, wherein the calculation unit includes a data acquisition control unit which acquires calculation data in which the result of the measurement by the blood vessel diameter measurement unit and the result of the acquisition by the blood pressure acquisition unit correspond to each other, and the calculation unit calculates the correlation, using the calculation data.

According to the second aspect of the invention, the correlation between the blood vessel diameter and the blood pressure can be calculated on the basis of the correspondence between the result of the measurement of the blood vessel diameter and the result of the acquisition of the blood pressure.

A third aspect of the invention is directed to the blood pressure measuring device according to the second aspect of the invention, wherein the data acquisition control unit acquires, as the calculation data, data in which a diastolic blood vessel diameter measured by the blood vessel diameter measurement unit and a diastolic blood pressure acquired by the blood pressure acquisition unit correspond to each other.

According to the third aspect of the invention, the correlation between the blood vessel diameter and the blood pressure can be calculated on the basis of the correspondence between the diastolic blood vessel diameter and the diastolic blood pressure.

A fourth aspect of the invention is directed to the blood pressure measuring device according to the second or third aspect of the invention, wherein the calculation unit includes a stability evaluation unit which evaluates stability of blood vessel diameter variation on the basis of the result of the measurement by the blood vessel diameter measurement unit, and the calculation unit calculates the correlation, using the calculation data in the case where a result of the evaluation by the stability evaluation unit satisfies a predetermined stability condition.

According to the fourth aspect of the invention, stability of blood vessel diameter variation can be evaluated on the basis of the result of the measurement of the blood vessel diameter, and the correlation between the blood vessel diameter and the blood pressure can be calculated, using the calculation data in the case where the blood vessel diameter variation is evaluated as stable. Therefore, a calculation with higher accuracy can be carried out.

A fifth aspect of the invention is directed to the blood pressure measuring device according to the fourth aspect of the invention, wherein the calculation unit causes the data acquisition control unit to acquire the calculation data if the result of the evaluation by the stability evaluation unit satisfies the stability condition.

According to the fifth aspect of the invention, acquisition of the calculation data only in the case where the blood vessel diameter variation is evaluated as stable is made possible.

A sixth aspect of the invention is directed to the blood pressure measuring device according to the fourth or fifth aspect of the invention, wherein the calculation unit stores the result of the evaluation by the stability evaluation unit based on the result of the measurement by the blood vessel diameter measurement unit, included in the calculation data, in association with the calculation data at each timing, selects calculation data to be used for calculation of the correlation from the calculation data at each timing on the basis of the result of the evaluation, and carries out the calculation.

According to the sixth aspect of the invention, calculation data to be used for calculation of the correlation between the blood vessel diameter and the blood pressure can be selected on the basis of the result of the evaluation based on the result of the measurement of the blood vessel diameter included in the calculation data.

A seventh aspect of the invention is directed to the blood pressure measuring device according to any of the first to sixth aspects of the invention, wherein the calculation unit carries out the calculation when a subject undergoes artificial dialysis.

According to the seventh aspect of the invention, the calculation can be carried out when the subject undergoes artificial dialysis. During artificial dialysis, change in blood pressure with time change is greater than in normal time. Therefore, it is anticipated that blood pressure values necessarily vary according to the lapse of time. Thus, significant data for the calculation of the interrelation can be obtained.

An eighth aspect of the invention is directed to the blood pressure measuring device according to any of the first to seventh aspects of the invention, wherein the blood pressure measuring device further includes a storage unit which stores the correlation as a lookup table of the blood vessel diameter of the blood vessel and the blood pressure in the blood vessel, and the blood pressure calculation unit calculates the blood pressure on the basis of the blood vessel diameter measured by the blood vessel diameter measurement unit, with reference to the lookup table.

According to the eighth aspect of the invention, the correlation between the blood vessel diameter and the blood pressure can be stored as a lookup table. Therefore, the calculation load in calculating the blood pressure can be reduced.

### BRIEF DESCRIPTION OF THE DRAWINGS

The invention will be described with reference to the accompanying drawings, wherein like numbers reference like elements.

FIG. 1 shows an example of application of a blood pressure measuring device.

FIG. 2 shows a correlation between blood vessel diameter and blood pressure.

FIG. 3 illustrates the calculation principle of a correlation formula expressing the correlation between blood vessel diameter and blood pressure.

FIG. 4 is a block diagram showing an example of the main functional configuration of the blood pressure measuring device.

FIG. 5 illustrates the principle of measurement of blood vessel diameter by a blood vessel diameter measurement unit.

FIG. 6 shows an example of the data configuration of measurement history for calculation.

FIG. 7 is a flowchart showing a processing procedure of calculation processing.

FIG. 8 is a graph showing an example of blood vessel diameter variation.

FIG. 9 shows an example of the overall configuration of a blood pressure measuring device according to a modification.

FIG. 10 is a flowchart showing a modification of the calculation processing.

FIG. 11 shows a modification of the correlation formula data.

### DESCRIPTION OF EXEMPLARY EMBODIMENTS

Hereinafter, an embodiment of an ultrasonic blood pressure measuring device and an ultrasonic blood pressure measuring method according to the invention will be described with reference to the drawings. Possible forms of application of the invention are not limited to the following embodiment. In the drawings, the same parts are denoted by the same reference numerals.

### Overall Configuration

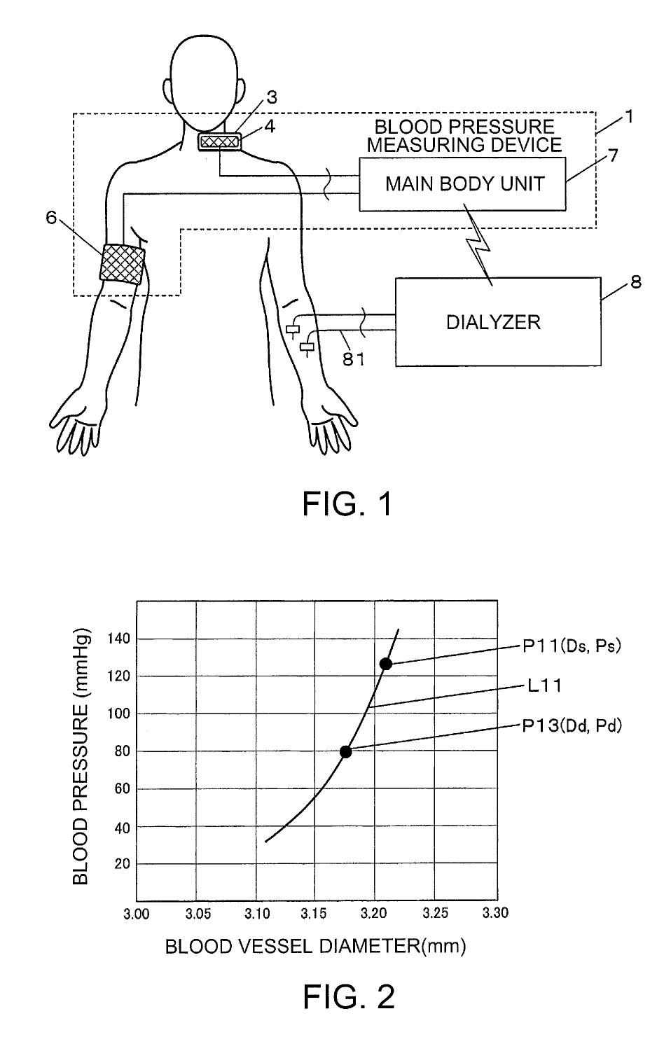

FIG. 1 shows an example of application of a blood pressure measuring device 1 according to this embodiment. The blood pressure measuring device 1 of this embodiment is configured as a combination of an ultrasonic blood pressure measuring device which measures blood pressure in a non-pressurizing manner by utilizing ultrasonic waves and a pressurizing-type sphygmomanometer. The blood pressure measuring device 1 includes an ultrasonic probe 3, a cuff 6, and a main body unit 7 as shown in FIG. 1.

That is, the blood pressure measuring device 1 is configured to find blood pressure in an estimating manner on the basis of the blood vessel diameter of a measurement target blood vessel (for example, carotid artery). The ultrasonic probe 3 has an ultrasonic sensor 4 to measure the blood vessel diameter via ultrasonic waves. To measure the blood vessel diameter, the ultrasonic probe 3 is attached, for example, to the subject's neck and the ultrasonic sensor 4 is positioned right above the carotid artery.

Also, to calculate blood pressure on the basis of the blood vessel diameter, blood pressure needs to be measured for calculation, apart from the blood vessel diameter. The cuff 6 is a pressurizing cuff to measure this blood pressure. The blood pressure measuring device 1 carries out pressurizing-type measurement of blood pressure, for example, using the cuff 6. FIG. 1 shows a type of cuff that is wound on the subject's upper arm to measure blood pressure in the upper arm artery. After the blood pressure measuring device 1 carries out calculation processing, the cuff 6 is removed from the subject. Subsequently, the blood pressure of the subject is measured in a non-pressurizing manner using the ultrasonic probe 3 only.

The main body unit 7 is connected to the ultrasonic sensor 4 of the ultrasonic probe 3 and to the cuff 6. The main body unit 7 carries out measurement of blood vessel diameter via ultrasonic waves using the ultrasonic probe 3 and pressurizing-type measurement of blood pressure using the cuff 6 and calculates the blood pressure of the subject. In this embodiment, it is assumed that the blood pressure measuring device 1 is used during dialysis treatment (artificial dialysis), and the main body unit 7 is configured to be able to transmit and receive data to and from a dialyzer 8 via wireless communication or the like. The dialyzer 8 is configured to circulate the blood of the subject (patient) through a dialysis tube 81 and carry out dialysis treatment while monitoring the blood pressure of the subject measured by the blood pressure measuring device 1. During dialysis treatment, blood pressure tends to change greatly with time change and tends to fall. Therefore, during dialysis treatment, the blood pressure of the subject is periodically measured for the purpose of monitoring situations where the blood pressure suddenly falls.

### Outline

To calculate blood pressure on the basis of the blood vessel diameter, the correlation between blood vessel diameter and blood pressure can be utilized. FIG. 2 shows the correlation between blood vessel diameter and blood pressure (hereinafter referred to as “correlation” or “interrelation” as needed). As shown by a curve L11 in FIG. 2, blood vessel diameter and blood pressure can be connected together as a certain nonlinear interrelation. This correlation between blood vessel diameter and blood pressure can be expressed by the following correlation formula (1) based on the pressure applied to the blood vessel and the blood vessel diameter at each blood pressure.

P=Pd−exp[β(D/Dd−1)] (1)

However, β=ln(Ps/Pd)/(Ds/Dd−1) (2)

In the above formula (1), “Ps” is the systolic blood pressure (maximum blood pressure) and “Pd” is the diastolic blood pressure (minimum blood pressure). “Ds” is the systolic blood vessel diameter, which is the blood vessel diameter at the systolic blood pressure, and “Dd” is the diastolic blood vessel diameter, which is the blood vessel diameter at the diastolic blood pressure. “β” is a blood vessel elasticity index value called stiffness parameter. In FIG. 2, the coordinate values defined by the systolic blood vessel diameter Ds and the systolic blood pressure Ps during a certain heartbeat are indicated by a plotted point p11, and the coordinates defined by the diastolic blood vessel diameter Dd and the diastolic blood pressure Pd are indicated by a plotted point P13.

If the stiffness parameter β can be found, the correlation formula (1) connecting the stiffness parameter with the blood vessel diameter D and blood pressure P can be defined. Traditionally, a calculation method by detecting pulsation, measuring the systolic blood pressure Ps, the systolic blood vessel diameter Ds, the diastolic blood pressure Pd and the diastolic blood vessel diameter Dd during one heartbeat, providing the respective values Ps, Pd, Ds, Dd for the formulas (1) and (2), and thus calculating the stiffness parameter β and the formula (1), is generally known. After the correlation formula (1) is found, the blood pressure P can be calculated on the basis of the blood vessel diameter D, which is constantly measured.

In contrast, this embodiment has the following features about the way the correlation formula (1) is found.

These features will be explained in order. First, the outline will be described. The blood pressure measuring device 1 carries out measurement of blood vessel diameter and pressurizing-type measurement of blood pressure in parallel with each other at each calculation data acquisition timing that is set at predetermined time intervals, and acquires the correspondence between blood vessel diameter and blood pressure, as calculation data. Then, the correlation formula (1) is calculated in an appropriate manner using the calculation data acquired at each calculation data acquisition timing, thus calculating the correlation between blood vessel diameter and blood pressure.

The calculation data acquisition timing is set at time intervals of 30 minutes or an hour. In this embodiment, measurement of blood pressure carried out periodically at predetermined time intervals of 30 minutes or an hour during dialysis treatment is used, and this measurement timing is set as the calculation data acquisition timing. During dialysis treatment, it is anticipated (or expected) that a change in the blood pressure is large and the blood pressure value differs at respective measurement timings. Therefore, providing a certain time interval enables easy designation of timings when different blood pressures are observed. Thus, a number of significant sampling data with different blood pressure values can be obtained. A highly accurate correlation formula can be calculated, and the correlation between blood vessel diameter and blood pressure can be calculated with high accuracy with respect to the subject in question.

In this embodiment, the measurement site for the measurement of blood vessel diameter that is contacted by the ultrasonic probe 3 is the neck, and the measurement site for the pressurizing-type measurement of blood pressure on which the cuff 6 is wound is the upper arm.

The following is an important piece of knowledge to realize this embodiment. That is, the difference in blood pressure at each site in the body is particularly large during a systolic period but small during a diastolic period. Therefore, in this embodiment, the correspondence between the diastolic blood vessel diameter Dd and the diastolic blood pressure Pd is extracted from the results of the measurement of blood vessel diameter and the pressurizing-type measurement of blood pressure carried out in parallel with each other at the calculation data acquisition timings, and is defined as calculation data. Then, the correlation formula (1) is calculated, using the correspondences (Dd1, Pd1), (Dd2, Pd2), . . . (Ddn, Pdn) between the diastolic blood vessel diameter Dd and the diastolic blood pressure Pd that are different at the respective calculation data acquisition timings. This enables reduction in calculation errors due to the difference between the measurement sites used for the measurement of blood vessel diameter and the pressurizing-type measurement of blood pressure.

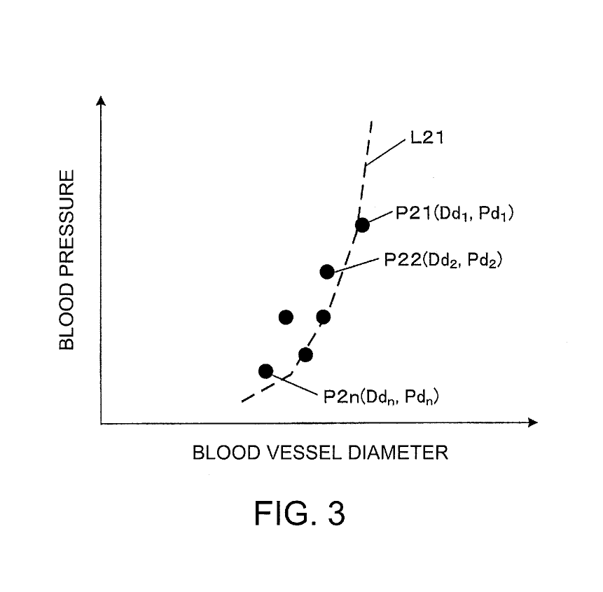

FIG. 3 illustrates the calculation principle of the correlation formula (1) and indicates the correspondences (Dd1, Pd1), (Dd2, Pd2), . . . (Ddn, Pdn) between the diastolic blood vessel diameter Dd and the diastolic blood pressure Pd acquired as calculation data, with plotted points P21, P22, . . . , P2n. In this embodiment, for example, the least squares method or the curve fitting processing or the like is used to approximate the correlation formula (1) with the correspondences (Dd1, Pd1), (Dd2, Pd2), . . . (Ddn, Pdn) of the respective plotted points P21, P22, . . . , P2n, thereby calculating the values of the respective parameters Pd, Dd, and β. Thus, the correlation formula (1) is calculated as an approximate curve L21 indicated by a dashed line in FIG. 3.

### Functional Configuration

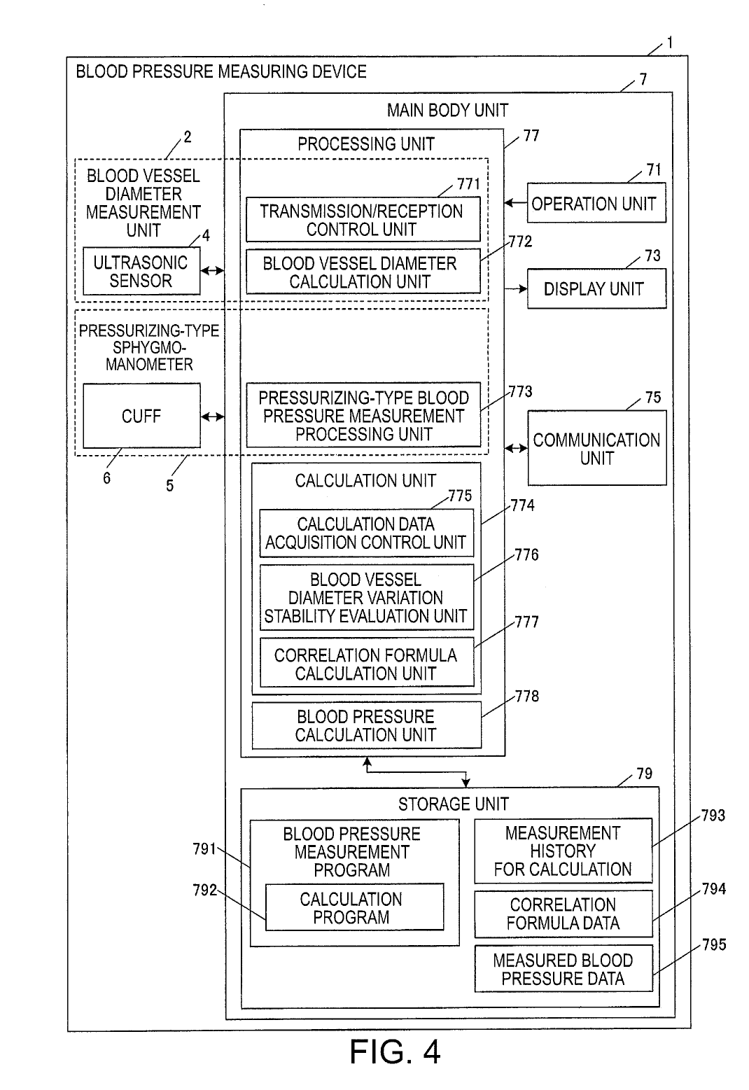

FIG. 4 is a block diagram showing an example of the main functional configuration of the blood pressure measuring device 1. The main body unit 7 of the blood pressure measuring device 1 includes an operation unit 71, a display unit 73, a communication unit 75, a processing unit 77, and a storage unit 79. The main body unit 7 is connected to the ultrasonic sensor 4 and the cuff 6.

The operation unit 71 is realized by an input device such as various switches including button switch, lever switch and dial switch, a touch panel, a track pad, and a mouse. The operation unit 71 outputs an operation signal corresponding to an operation input, to the processing unit 77.

The display unit 53 is realized by a display device such as an LCD (liquid crystal display) or EL display (electroluminescence display). The display unit 73 displays various screens based on display signals inputted from the processing unit 77. The measured blood pressure or the like of the subject is displayed on the display unit 73. For example, a current blood pressure display screen, or a blood pressure change display screen or the like in the form of a graph showing change in blood pressure based on logged data in the past, is displayed according to a display mode switch operation on the operation unit 71.

The communication unit 75 is communication device for transmitting and receiving data to and from outside (for example, the dialyzer 8), under the control of the processing unit 77. As a communication system of this communication unit 75, various systems can be applied such as a format in which wired connection is established via a cable conforming to a predetermined communication standard, a format in which connection is established via an intermediate device called cradle or the like that also functions as a charger, or a format in which wireless communication is used to establish wireless connection.

The processing unit 77 is a control device and arithmetic device which performs overall control over each part of the blood pressure measuring device 1. The processing unit is realized by a microprocessor such as CPU (central processing unit) or GPU (graphic processing unit), and an ASIC (application specific integrated circuit) or IC (integrated circuit) memory, or the like. The processing unit 77 includes a transmission/reception control unit 771, a blood vessel diameter calculation unit 772, a pressurizing-type blood pressure measurement processing unit 773, a calculation unit 774, and a blood pressure calculation unit 778. Each part forming the processing unit 77 may be configured as hardware.

The transmission/reception control unit 771 controls transmission and reception of ultrasonic waves by the ultrasonic sensor 4. Specifically, the transmission/reception control unit 771 outputs a transmission/reception control signal to the ultrasonic sensor 4 and performs control to switch between a transmission mode and a reception mode. The blood vessel diameter calculation unit 772 calculates a blood vessel diameter of a measurement target blood vessel on the basis of a result of signal processing inputted from the ultrasonic sensor 4.

The transmission/reception control unit 771 and the blood vessel diameter calculation unit 772, together with the ultrasonic sensor 4, form a blood vessel diameter measurement unit 2. The blood vessel diameter measurement unit 2 realizes measurement of blood vessel diameter.

Here, the ultrasonic sensor 4 is an ultrasonic wave transmission/reception unit and is formed by an ultrasonic wave transmitting/reception circuit. The transmission/reception circuit transmits and receives ultrasonic waves while switching between the transmission mode and the reception mode according to a transmission/reception control signal inputted from the transmission/reception control unit 771. Specifically, the transmission/reception circuit includes an ultrasonic oscillation circuit which generates a pulse signal with a predetermined frequency, a transmission delay circuit which delays the generated pulse signal, and the like, as a configuration for transmission. The transmission/reception circuit also includes a reception delay circuit which delays a received signal, a filter which extracts predetermined frequency component from the received signal, and an amplifier which amplifies the received signal, and the like, as a configuration for reception.

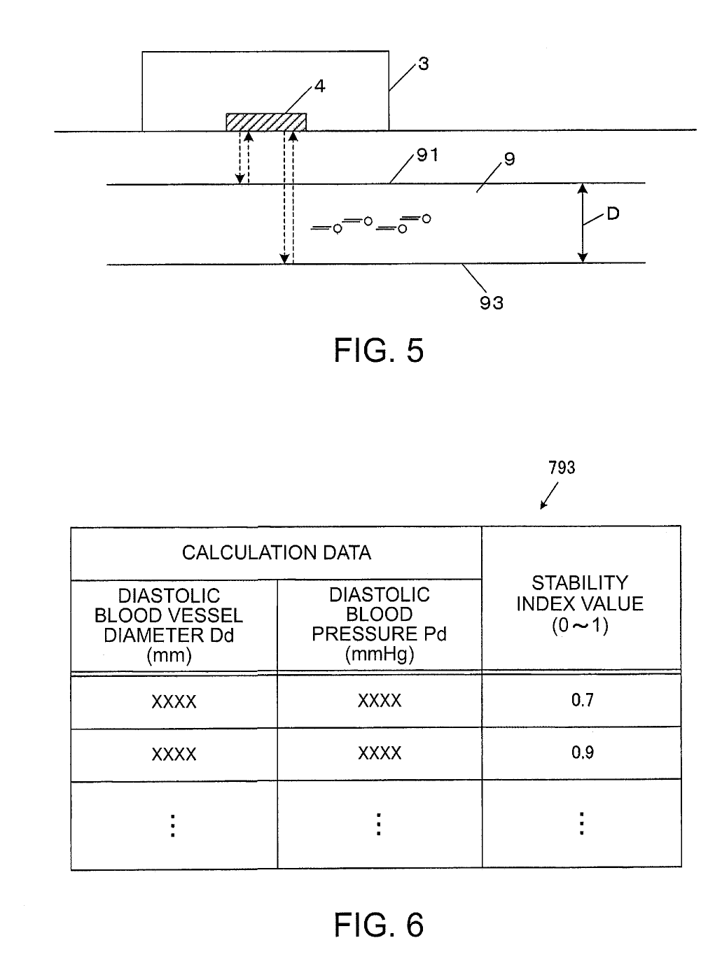

FIG. 5 illustrates the principle of the measurement of blood vessel diameter by the blood vessel diameter measurement unit 2. As described above, the ultrasonic sensor 4 is positioned right above a carotid artery 9 as the ultrasonic probe 3 is made to contact the subject's neck. The ultrasonic sensor 4 transmits an ultrasonic pulse signal or burst signal of several MHz to several ten MHz toward the carotid artery 9 and receives a reflected wave from a front wall 91 of the carotid artery 9 and a reflected wave from a rear wall 93, as indicated by dashed lines with arrows in FIG. 5. The reflected wave from the front wall 91 and the reflected wave from the rear wall 93, thus received, are amplified and signal-processed and subsequently outputted to the blood vessel diameter calculation unit 772. The blood vessel diameter calculation unit 772 calculates a blood vessel diameter D of the carotid artery 9 on the basis of the difference in reception time between the reflected wave from the front wall 91 and the reflected wave from the rear wall 93. By carrying out this measurement of blood vessel diameter continuously, it is possible to detect a difference in blood vessel diameter AD of the carotid artery 9 that varies with heartbeats.

The pressurizing-type blood pressure measurement processing unit 773 detects a pressure pulse wave while adjusting the pressure inside the cuff 6 and carries out processing to calculate a systolic blood pressure Ps and a diastolic blood pressure Pd, for example, by an oscillometric method, as a blood pressure acquisition unit which acquires the blood pressure of the subject. The pressurizing-type blood pressure measurement processing unit 773, together with the cuff 6, forms a pressurizing-type sphygmomanometer (cuff-type sphygmomanometer) 5. The pressurizing-type measurement of blood pressure is realized by the pressurizing-type sphygmomanometer 5.

The calculation unit 774 carries out processing to calculate the correlation between blood vessel diameter and blood pressure (calculation processing). The calculation unit 774 includes a calculation data acquisition control unit 775 as a data acquisition control unit, a blood vessel diameter variation stability evaluation unit 776 as a stability evaluation unit, and a correlation formula calculation unit 777.

The calculation data acquisition control unit 775 performs control to achieve execution of the measurement by the blood vessel diameter measurement unit 2 and execution of the measurement by the pressurizing-type sphygmomanometer 5 in parallel with each other, so that the measurement of blood vessel diameter and the pressurizing-type measurement of blood pressure are carried out in parallel.

The blood vessel diameter variation stability evaluation unit 776 controls continuous execution of the measurement of blood vessel diameter by the blood vessel diameter measurement unit 2 and evaluates stability of blood vessel diameter variation on the basis of the result of the continuous measurement, prior to the parallel execution control of the measurement of blood vessel diameter and the pressurizing-type measurement of blood pressure by the calculation data acquisition control unit 775.

The correlation formula calculation unit 777 calculates the correlation formula (1) on the basis of the result of the measurement by the blood vessel diameter measurement unit 2 and the result of the measurement by the pressurizing-type sphygmomanometer 5.

The blood pressure calculation unit 778 calculates blood pressure on the basis of the blood vessel diameter measured by the blood vessel diameter measurement unit 2, using the correlation formula (1) calculated by the correlation formula calculation unit 777. Thus, the blood pressure of the subject is measured in a non-pressurizing manner.

The storage unit 79 is realized by a storage medium such as IC memory, hard disk, or optical disc. In the storage unit 79, a program which causes the blood pressure measuring device 1 to operate and realize various functions of the blood pressure measuring device 1, and data or the like used during the execution of the program are stored in advance, or temporarily stored every time processing is carried out.

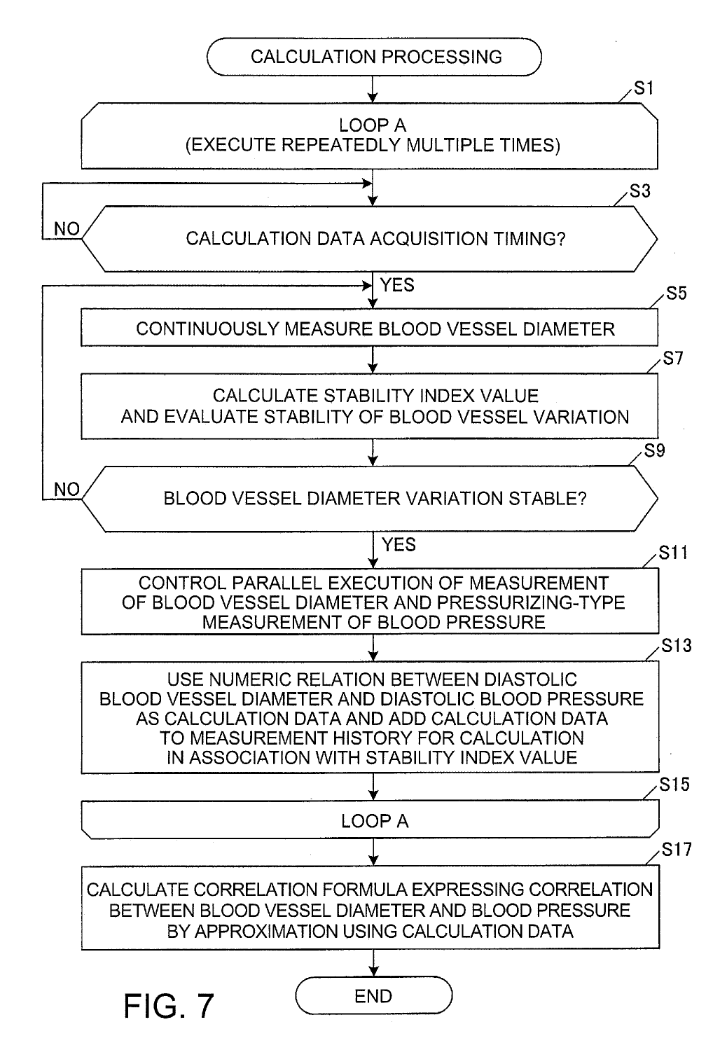

In the storage unit 79, a blood pressure measurement program 791 which causes the processing unit 77 to function as the transmission/reception control unit 771, the blood vessel diameter calculation unit 772, the pressurizing-type blood pressure measurement processing unit 773, the calculation unit 774 and the blood pressure calculation unit 778 is stored. The blood pressure measurement program 791 includes a calculation program 792 for executing calculation processing (see FIG. 7).

Also, a measurement history for calculation 793, correlation formula data 794 and measured blood pressure data 795 are stored as data in the storage unit 79.

In the measurement history for calculation 793, calculation data acquired by the calculation data acquisition control unit 775 to calculate the correlation formula are accumulated and stored. FIG. 6 shows an example of the data configuration of the measurement history for calculation 793. As shown in FIG. 6, the measurement history for calculation 793 is a data table in which the diastolic blood vessel diameter Dd and the diastolic blood pressure Pd, acquired as calculation data by carrying out the measurement of blood vessel diameter and the pressurizing-type measurement of blood pressure in parallel at each calculation data acquisition timing, correspond to a stability index value. The stability index value is calculated by the blood vessel diameter variation stability evaluation unit 776 in order to evaluate stability of blood vessel diameter variation, when acquiring the corresponding calculation data.

The correlation formula data 794 stores the values of the respective parameters Pd, Dd, and β in the formula (1) for each subject, as data of the correlation formula expressing the correlation between blood vessel diameter and blood pressure calculated by the correlation formula calculation unit 777.

The measured blood pressure data 795 stores the blood pressure calculated at each measurement timing by the blood pressure calculation unit 778.

### Processing Flow

FIG. 7 is a flowchart showing the processing procedure of calculation processing. The processing described here can be realized as the calculation unit 774 reads out and executes the calculation program 792 from the storage unit 79. The blood pressure measuring device 1 carries out calculation processing by carrying out processing according to the processing procedure of FIG. 7. After this processing, the blood pressure measuring device 1 carries out measurement of blood vessel diameter via ultrasonic waves without using the cuff 6, and carries out ultrasonic measurement of blood pressure in which blood pressure is calculated in an estimating manner. Therefore, the cuff 6 can be removed after the calculation processing.

As shown in FIG. 7, in the calculation processing, the calculation unit 774 first repeatedly executes processing of a loop A (Steps S1 to S15). In this embodiment, as the processing of the loop A is repeated until one session of dialysis treatment ends, calculation data is acquired utilizing the measurement of blood pressure during the one session of dialysis treatment.

In the loop A, a standby state is maintained until the present time reaches a preset calculation data acquisition timing (Step S3: NO). In this embodiment, the time when the dialysis treatment starts and the timing of measurement of blood pressure that is carried out periodically during the dialysis treatment are used as calculation data acquisition timings. Also, data of the elapsed time of the dialysis treatment may be received from the dialyzer 9 via the communication unit 75, and whether the present time is a calculation data acquisition timing or not may be determined. Then, the processing unit 77 executes the processing of Step S5 and onward every time the calculation data acquisition timing, that is, the measurement timing is reached (Step S3: YES).

That is, first, the blood vessel diameter variation stability evaluation unit 776 controls continuous execution of the measurement of blood vessel diameter by the blood vessel diameter measurement unit 2 and thus continuously carries out the measurement of blood vessel diameter during a continuous measurement period of several seconds to several ten seconds (Step S5). This processing can be realized, for example, by applying a known technique such as phase difference tracking.

Next, the blood vessel diameter variation stability evaluation unit 776 evaluates stability of blood vessel diameter variation in a diastolic period on the basis of the blood vessel diameter measured continuously in Step S5 (Step S7).

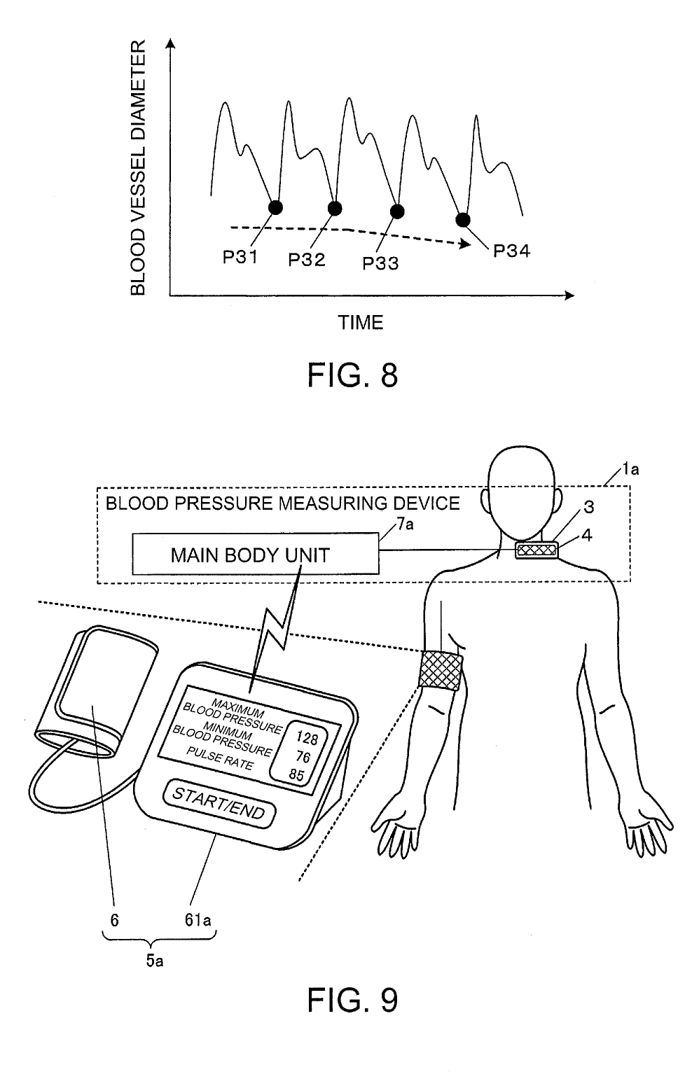

FIG. 8 is a graph showing an example of change in blood vessel diameter over time (blood vessel diameter variation) measured continuously in Step S5, in which the diastolic blood vessel diameter Dd is indicated by black plotted points P31 to P34. As shown in FIG. 8, the blood vessel diameter during the continuous measurement period gradually decreases as a whole with a fall in blood pressure during the dialysis treatment, and the diastolic blood vessel diameter Dd at each heartbeat tends to decrease. However, during dialysis treatment, blood pressure can suddenly fall and therefore the blood vessel diameter can suddenly decrease. Calculation data that is acquired when the blood vessel diameter variation is thus large and unstable, is more likely to have an error at the time of calculation than calculation data that is acquired when the blood vessel diameter variation is small and stable. In order to reduce this error, the blood vessel diameter variation stability evaluation unit 776 evaluates the blood vessel diameter variation on the basis of each value of the diastolic blood vessel diameter Dd during the continuous measurement period indicated by the plotted points P31 to P34. Specifically, the blood vessel diameter variation stability evaluation unit 776 extracts the diastolic blood vessel diameter Dd from the result of the continuous measurement of blood vessel diameter, and determines whether blood pressure is stable or not, on the basis of the degree of fluctuation thereof.

For example, the degree of fluctuation of the amount of variation in the blood vessel diameter per heartbeat within the continuous measurement period (difference between the systolic blood vessel diameter Ds and the diastolic blood vessel diameter Dd) (for example, it may be a standard deviation or average value) and the degree of fluctuation of the amount of variation in the diastolic blood vessel diameter Dd (for example, a standard deviation or difference between maximum value and minimum value of the diastolic blood vessel diameter Dd within the continuous measurement period, or the like) are calculated. The average of these degrees of fluctuation or the larger one of these is selected. Then, a stability index value is determined, for example, within a range of 0 to 1 in such a way that the stability index value becomes greater as the selected degree becomes lower, whereas the stability index value becomes smaller as the selected degree becomes higher. Also, for example, a threshold (for example, 0.7) for the stability index value is set in advance according to what degree the fluctuation of the diastolic blood vessel diameter Dd is tolerated with respect to the average amount of variation in the blood vessel diameter per heartbeat. Then, whether the blood vessel diameter variation is stable or unstable is determined, for example, using a stability condition that the stability index value thus found exceeds the threshold. Thus, for example, if the amount of variation in the blood vessel diameter per heartbeat is approximately 400 μm or smaller and the fluctuation of the diastolic blood vessel diameter Dd is approximately 40 μm or smaller, it is possible to determine that the blood vessel diameter variation is stable.

Next, the blood vessel diameter variation stability evaluation unit 776 performs threshold processing on the stability index value calculated in Step S7. If the stability index value exceeds a predetermined threshold (for example, 0.7), the blood vessel diameter variation stability evaluation unit 776 determines that the blood vessel diameter variation is stable (Step S9: YES) and shifts to Step S11. Meanwhile, if the stability index value is equal to or below the predetermined threshold, the blood vessel diameter variation stability evaluation unit 776 determines that the blood vessel diameter variation is unstable (Step S9: NO) and returns to Step S5 to repeat the foregoing processing.

Subsequently, in Step S11, the calculation data acquisition control unit 775 performs control to achieve execution of the measurement by the blood vessel diameter measurement unit 2 and execution of the measurement by the pressurizing-type sphygmomanometer 5 in parallel with each other. Then, the calculation data acquisition control unit 775 extracts each value of the diastolic blood vessel diameter Dd and the diastolic blood pressure Pd from the results of the measurements in Step S11, as calculation data, and adds the calculation data to the measurement history for calculation 793 in association with the stability index value calculated in Step S7 (Step S13).

Repeating the above processing of the loop A as one session of acquisition of calculation data during the dialysis treatment enables acquisition of plural calculation data. As the processing of the loop A is finished, the correlation formula calculation unit 777 calculates the correlation formula (1) by approximation using the correspondence between the plural diastolic blood vessel diameters Dd and diastolic blood pressures Pd acquired as the calculation data, with reference to the measurement history for calculation 793 (Step S17). The data of the calculated correlation formula (the values of the respective parameters Ps, Ds, and β in the formula (1)) are stored as the correlation formula data 794 in the storage unit 79. The processing then ends.

After the calculation processing is thus carried out (for example, during the next session of dialysis treatment and onward), only the measurement of blood vessel diameter is carried out in the measurement of the blood pressure of the subject. Then, the blood pressure calculation unit 778 calculates blood pressure from the blood vessel diameter that is measured, according to the correlation formula expressing correlation between the blood vessel diameter and the blood pressure of the subject in question stored as the correlation formula data 794. Prior to this processing, stability of blood vessel diameter variation may be evaluated by a technique similar to Steps S5 to S9 in FIG. 7. Then, immediately after blood vessel diameter variation is determined as stable, the blood vessel diameter may be measured to calculate blood pressure. Thus, if blood vessel diameter variation is determined as unstable, continuous measurement of blood vessel diameter may be repeated until blood vessel diameter variation is determined as stable, and the actual timing of measuring blood pressure can thus be adjusted to the timing when blood vessel diameter variation is stable. The blood pressure thus calculated is displayed on the display unit 73 and is also transmitted to the dialyzer 8 via the communication unit 75 so as to be used for dialysis treatment.

The correlation formula (1) can change according to change in the stiffness of the subject's blood vessel. In such a case, the correlation formula needs to be recalculated to recalculate the correlation between blood vessel diameter and blood pressure. However, since the stiffness of blood vessel generally does not change suddenly, once the calculation is carried out, there is no need to subsequently calculate the correlation over a relative long period such as several months. Therefore, for example, during dialysis treatment for that period, simply bringing the ultrasonic probe 3 in contact with the subject's neck enables measurement of blood pressure. Thus, the burden on the subject can be reduced and the time and effort for the calculation can be reduced as well.

As described above, according to the embodiment, the timing of measuring blood pressure measured periodically during dialysis treatment can be used as a calculation data acquisition timing, to acquire calculation data in which the diastolic blood vessel diameter Dd and the diastolic blood pressure Pd correspond to each other at each calculation data acquisition timing. Calculating the correlation formula expressing the correlation between blood vessel diameter and blood pressure by approximation using the acquired calculation data enables calculation of the correlation between blood vessel diameter and blood pressure. Therefore, the burden on the subject due to the calculation and the time and effort for the calculation can be reduced. Also, since the measurement timing during dialysis treatment, in which it is anticipated (or expected) that change in blood pressure with time change is larger than in normal time, resulting in different blood pressure values, can be used as a calculation data acquisition timing to acquire calculation data, plural sampling data that are significant for the calculation of the correlation between blood vessel diameter and blood pressure can be acquired and the accuracy of the calculation can be improved. Consequently, blood pressure can be accurately calculated on the basis of the blood diameter vessel and accuracy of non-pressurizing-type measurement of the subject's blood pressure can be improved.

Moreover, the blood vessel diameter can be continuously measured to evaluate stability immediately before calculation data is acquired. If blood vessel diameter variation is evaluated as unstable, the continuous measurement of blood vessel diameter is carried out repeatedly until blood vessel diameter variation is evaluated as stable. Thus, the actual timing of acquiring calculation data can be adjusted to the timing when blood vessel diameter variation is stable. This enables calculation of the correlation between blood vessel diameter and blood pressure, using the correspondence between the diastolic blood vessel diameter Dd and the diastolic blood pressure Pd acquired when blood vessel diameter variation is stable. Therefore, calculation error in the correlation between blood vessel diameter and blood pressure can be reduced and accuracy of the calculation can be improved further. Also, since calculation data is not acquired at a timing when blood vessel diameter variation is unstable, useless pressurizing-type measurement of blood pressure is not carried out. This can reduce the burden on the subject.

Also, the correspondence between the diastolic blood vessel diameter Dd and the diastolic blood pressure Pd during a diastolic period, in which the difference in blood pressure at each site in the body is small, is acquired as calculation data. Therefore, accuracy of the calculation of the correlation formula between blood vessel diameter and blood pressure can be improved further, realizing further improvement in calculation accuracy.

In the embodiment, the pressurizing-type sphygmomanometer 5 using an oscillometric method is employed as an example of the configuration for measuring blood pressure. However, the configuration for measuring blood pressure is not particularly limited. For example, a blood pressure meter using a tonometry method or a volume compensation method, which is a type of continuous method, to measure blood pressure, or a blood pressure meter using an auscultatory method (Korotkoff method), which is a type of intermittent method, to measure blood pressure, or the like may be employed according to need.

The configuration of the blood pressure measuring device 1 is not limited to the configuration shown in FIG. 1. For example, the blood pressure measuring device may be configured with the pressurizing-type sphygmomanometer 5 shown in FIG. 1 as a separate unit. FIG. 9 shows an example of the configuration of a blood pressure measuring device 1a according to this modification. The blood pressure measuring device 1a shown in FIG. 9 is configured in such a way that a main body unit 7a of the blood pressure measuring device 1a and a main body unit 61a of a pressurizing-type sphygmomanometer 5a are capable of transmitting and receiving data between each other, and the blood pressure measuring device 1a acquires blood pressure values from the pressurizing-type sphygmomanometer 5a. This blood pressure measuring device 1a can be realized by separating the pressurizing-type sphygmomanometer 5 from the blood pressure measuring device 1 shown in FIG. 1. The main body unit 7a of the blood pressure measuring device 1a acquires and uses the result of pressurizing-type measurement of blood pressure by the pressurizing-type sphygmomanometer 5a, to calculate the correlation formula (1) in a similar manner to the above embodiment, thus calculating the correlation between blood vessel diameter and blood pressure. Although not shown in FIG. 9, the main body unit 7a of the blood pressure measuring device 1a may be connected for communication with the dialyzer 8 shown in FIG. 1 according to need. According to this modification, the correlation between blood vessel diameter and blood pressure can be calculated, using blood pressure measured by an external blood pressure meter such as pressurizing-type sphygmomanometer.

Also, the site for measuring blood pressure by a blood pressure meter such as a pressurizing-type sphygmomanometer is not limited to the illustrated upper arm and may be another site such as the wrist.

In the embodiment, the stability index value is calculated on the basis of the result of continuous measurement of blood vessel diameter, thus evaluating stability of blood vessel diameter variation. However, this evaluation technique is not limiting. For example, measurement of blood vessel diameter and pressurizing-type measurement of blood pressure may be carried out in parallel immediately before the calculation processing is started, so as to measure the systolic blood pressure Ps and the systolic blood vessel diameter Ds, and the diastolic blood pressure Pd and the diastolic blood vessel diameter Dd, during a heartbeat. Then, the correlation formula (1) may be calculated for evaluation, using a traditional blood pressure calculation method with the use of the stiffness parameter β. Then, blood pressure may be calculated on the basis of the result of continuous measurement of blood vessel diameter, using the correlation formula for evaluation. If change in blood pressure within the continuous measurement period is equal to or below a predetermined value (for example, 5 mmHg) that is set in advance, blood vessel diameter variation may be evaluated as stable. If change in blood pressure is above the predetermined value, blood vessel diameter variation may be evaluated as unstable. In the case where blood vessel diameter variation is evaluated as stable, measurement of blood vessel diameter and pressurizing-type measurement of blood pressure may be carried out in parallel to acquire calculation data.

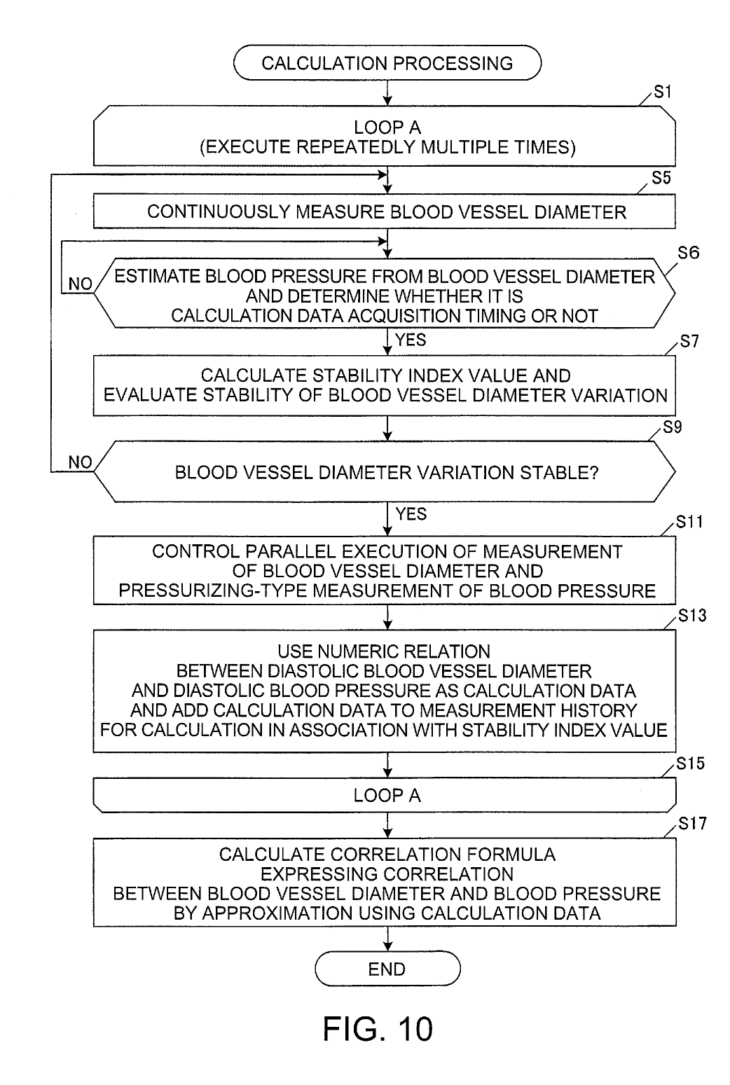

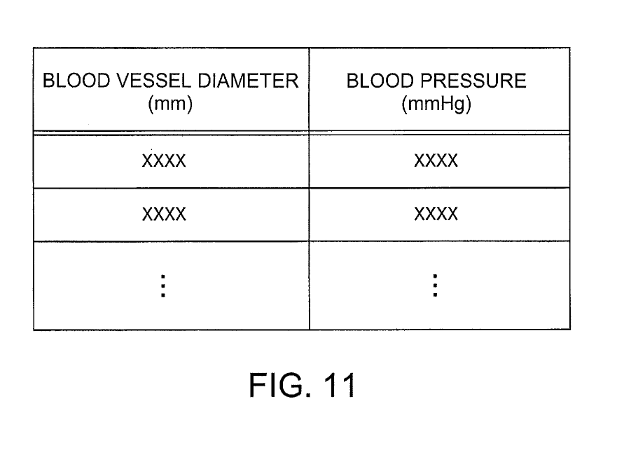

In the embodiment, the calculation data acquisition timing is described as being determined by time (for example, at predetermined time intervals during dialysis treatment). However, other configurations may also be employed. For example, a timing when a predetermined condition is met, indicating that the amount of variation in blood vessel diameter or blood pressure becomes large, may be used as the calculation data acquisition timing. More specifically, measurement of blood vessel diameter and pressurizing-type measurement of blood pressure may be carried out in parallel immediately before the calculation processing is started, so as to measure the systolic blood pressure Ps and the systolic blood vessel diameter Ds, and the diastolic blood pressure Pd and the diastolic blood vessel diameter Dd, during a heartbeat. Then, the correlation formula (1) may be calculated for timing determination, using a traditional blood pressure calculation method with the use of the stiffness parameter β. Then, the calculation processing is executed according to the flowchart of FIG. 10. In the flowchart of FIG. 10, Step S3 in FIG. 7 is deleted and new processing of Step S6 is added between Steps S5 and S7. That is, blood pressure may be estimated on the basis of the blood vessel diameter continuously measured in Step S5 and the correlation formula for timing determination calculated in advance. Subsequently, if a predetermined condition is met, indicating that the amount of variation in the estimated blood pressure becomes large, this timing is determined as calculation data acquisition timing (Step S6).

In the embodiment, threshold processing is performed on the stability index value calculated on the basis of the result of continuous measurement of blood vessel diameter. In the case where the stability index value is equal to or above a predetermined threshold that is set in advance and therefore blood vessel diameter variation is determined as stable, calculation data is acquired. In contrast, while the stability index value is calculated, calculation data may be acquired without determining whether blood vessel diameter variation is stable or not, and the calculation data may be stored corresponding to the calculated stability index value. When calculating the correlation formula (1), calculation data corresponding to the stability index value equal to or above a predetermined threshold may be selected and used from the respective calculation data.

In the embodiment, calculation data is acquired, using all the measurement timing during one session of dialysis treatment as calculation data acquisition timings, and the correlation formula (1) is calculated to calculate the correlation between blood vessel diameter and blood pressure. Meanwhile, the first plural measurement timings following the start of dialysis treatment may be used as calculation data acquisition timing to acquire calculation data. In this case, the number of times calculation data is acquired may be set in advance and the processing of the loop A in FIG. 7 may be repeated simply the number of times of acquisition. The reduction in the number of times of acquisition of calculation data leads to reduction in the number of times of pressurizing-type measurement of blood pressure for calculation. Therefore, the burden on the subject due to the calculation can be reduced further. As the number of times of acquisition of calculation data increases, the correlation formula (1) can be approximated with higher precision in the subsequent processing. However, the acquisition of calculation data may be carried out at least twice.

In the embodiment, all the acquired calculation data are used to calculate the correlation formula (1). Meanwhile, a part of the acquired calculation data may be used to calculate the correlation formula (1). For example, the acquired calculation data may be rearranged in order from the largest stability index value, and a predetermined number of (for example, five) calculation data from the top may be selected and used to calculate the correlation formula (1).

In the embodiment, calculation data is acquired, using change in blood pressure during dialysis treatment in order to calculate the correlation between blood vessel diameter and blood pressure. However, the situation where calculation data is acquired is not limited to during dialysis treatment. For example, change in blood pressure due to exercise may be used, and plural timings including at-rest time and during exercise may be used as calculation data acquisition timings to acquire calculation data.

Also, processing to transmit the calculation data accumulated as the measurement history for calculation 793 to an external device, for example, a smartphone or server, via the communication unit 75 may be carried out, so that the calculation data may be managed in the external device.

In the embodiment, the correlation formula data 794 is described as data storing the value of each parameter in the formula (1). However, different formats may also be employed. For example, after the value of each parameter is found and the formula (1) is thus derived, a lookup table as shown in FIG. 11 that defines the correspondence between blood vessel diameter and blood pressure on the basis of the formula (1) may be found and used as the correlation formula data 794. The space between blood vessel diameters employed in the lookup table may be arbitrarily determined, for example, every several μm to several ten μm.

The blood pressure calculation unit 778 can calculate blood pressure on the basis of the blood vessel diameter measured by the blood vessel diameter measurement unit 2, with reference to the lookup table. Thus, the arithmetic load on the blood pressure calculation unit 778 to calculate blood pressure can be reduced.

The entire disclosure of Japanese Patent Applications Nos. 2013-219893, filed Oct. 23, 2013, and 2014-151559, filed Jul. 25, 2014, are expressly incorporated by reference herein.

What is claimed is:

1. A blood pressure measuring device comprising:
a blood vessel diameter measurement unit which measures a blood vessel diameter of a blood vessel;
a blood pressure acquisition unit which acquires a blood pressure in the blood vessel;
a calculation unit which calculates a correlation between the blood vessel diameter of the blood vessel and the blood pressure in the blood vessel on the basis of a result of the measurement by the blood vessel diameter measurement unit and a result of the acquisition by the blood pressure acquisition unit; and
a blood pressure calculation unit which calculates a blood pressure in the blood vessel on the basis of the blood vessel diameter of the blood vessel measured by the blood vessel diameter measurement unit, using the correlation.

2. The blood pressure measuring device according to claim 1, wherein the calculation unit includes a data acquisition control unit which acquires calculation data in which the result of the measurement by the blood vessel diameter measurement unit and the result of the acquisition by the blood pressure acquisition unit correspond to each other, and the calculation unit calculates the correlation, using the calculation data.

3. The blood pressure measuring device according to claim 2, wherein the data acquisition control unit acquires, as the calculation data, data in which a diastolic blood vessel diameter measured by the blood vessel diameter measurement unit and a diastolic blood pressure acquired by the blood pressure acquisition unit correspond to each other.

4. The blood pressure measuring device according to claim 2, wherein the calculation unit includes a stability evaluation unit which evaluates stability of blood vessel diameter variation on the basis of the result of the measurement by the blood vessel diameter measurement unit, and the calculation unit calculates the correlation, using the calculation data in the case where a result of the evaluation by the stability evaluation unit satisfies a predetermined stability condition.

5. The blood pressure measuring device according to claim 4, wherein the calculation unit causes the data acquisition control unit to acquire the calculation data if the result of the evaluation by the stability evaluation unit satisfies the stability condition.

6. The blood pressure measuring device according to claim 4, wherein the calculation unit stores the result of the evaluation by the stability evaluation unit based on the result of the measurement by the blood vessel diameter measurement unit, included in the calculation data, in association with the calculation data at each timing, selects calculation data to be used for calculation of the correlation from the calculation data at each timing on the basis of the result of the evaluation, and carries out the calculation.

7. The blood pressure measuring device according to claim 1, wherein the calculation unit carries out the calculation when a subject undergoes artificial dialysis.

8. The blood pressure measuring device according to claim 1, further comprising a storage unit which stores the correlation as a lookup table of the blood vessel diameter of the blood vessel and the blood pressure in the blood vessel,
wherein the blood pressure calculation unit calculates the blood pressure on the basis of the blood vessel diameter measured by the blood vessel diameter measurement unit, with reference to the lookup table.

9. A blood pressure measuring method comprising:
carrying out measurement of a blood vessel diameter of a blood vessel and acquisition of a blood pressure in the blood vessel;
calculating a correlation between the blood vessel diameter of the blood vessel and the blood pressure in the blood vessel on the basis of a result of the measurement of the blood vessel diameter and a result of the acquisition of the blood pressure; and
calculating a blood pressure in the blood vessel on the basis of the result of the measurement of the blood vessel diameter, using the correlation.
